(19) United States
(12) Patent Application Publication
(10) Pub. No.: US 2018/0071391 A9
(43) Pub. Date: Mar. 15, 2018

(54) Topical Pharmaceutical Cosmetic and Disinfectant Compositions Comprising Phosphatidylcholine

(71) Applicant: LIPIDOR AB, Stockholm (SE)

(72) Inventors: Bengt Herslöf, Stockholm (SE); Jan Holmbäck, Vaxholm (SE)

(73) Assignee: LIPIDOR AB, Stockholm (SE)

(21) Appl. No.: 15/036,567

(22) PCT Filed: Nov. 6, 2014
(86) PCT No.: PCT/SE2014/051314

§ 371 Date: May 13, 2016

Related U.S. Application Data
(63) Continuation-in-part of application No. 14/114,778, filed on Oct. 30, 2013, now abandoned.

(30) Foreign Application Priority Data
May 2, 2011 (SE) 1100340-7
Nov. 14, 2013 (SE) 1300709-1

(51) Int. Cl. A61K 47/24; A61K 47/02; A61K 31/14; A61K 31/235; A61K 31/573; A61K 31/155; A61K 31/196; A61K 31/565; A61K 31/506; A61K 31/351; A61K 31/436; A61K 31/4164; A61K 38/08; A61K 31/167; A61K 31/047; A61K 31/7036; A61K 8/58; A61K 8/55; A61Q 17/00; A61Q 19/00; A61Q 17/04

(52) U.S. Cl. CPC A61K 47/24; A61Q 17/04; A61K 31/14; A61K 31/235; A61K 31/573; A61K 31/155; A61K 31/196; A61K 31/565; A61K 31/506; A61K 31/351; A61K 31/436; A61K 31/4164; A61K 38/08; A61K 31/167; A61K 31/047; A61K 31/7036; A61K 8/585; A61K 8/553; A61Q 17/005; A61Q 19/00; A61K 47/02

(57) ABSTRACT

A topical carrier consists of 99% by weight or more of phosphatidylcholine and volatile solvent selected from the group consisting of: ethanol and its combinations with C3-and/or C4-alcohol and/or volatile silicone oil. The carrier may additionally comprise up to 1% by weight of antioxidant, colorant, odorant, and/or preservative, and up to 2% by weight of denaturant. Also disclosed are pharmaceutical, cosmetic and disinfectant compositions consisting of the carrier and pharmaceutically active agent, cosmetically active agent and/or disinfectant agent.

### FIELD OF THE INVENTION

The present invention relates to a topical pharmaceutical, a cosmetic, and/or a disinfectant composition. The present invention furthermore relates to a corresponding carrier.

### BACKGROUND OF THE INVENTION

Pharmaceutical compositions for topical administration are of two kinds: one kind aiming at administering a pharmaceutically active agent onto healthy or diseased skin to produce its effect on the skin and/or in one or more layers of the skin, the other kind aiming at the delivery of a pharmaceutically active agent through the skin. Cosmetic compositions are related to the first kind since they are specifically designed for producing their effect on the skin. In this application “on the skin” includes its outermost layer, the stratum corneum. Disinfectant compositions are also related to the first kind since they are designed to produce their effect on the skin. A disinfectant composition destroys or at least inhibits the growth of harmful organisms on the skin.

For topical compositions of the first kind it is important to increase the water content of the stratum corneum, to supply lipid-like substances to enhance the barrier function of the skin, and to improve lubricating properties between keratin units (Larsson K et al., Lipids—Structure, Physical Properties and Functionality. Oily Press Ltd, 2006, p. 149-153).

U.S. Pat. No. 6824785 B1 discloses a transdermal water loss-reducing topical composition containing an aqueous dispersion of at least two lipids in a non-crystalline phase lamellar array. The composition can be formulated as a pharmaceutical preparation. After administration the dried composition adopts a crystalline lamellar phase on the skin.

U.S. Pat. No. 8147833 B1 discloses a method of treating skin conditions such as atopic dermatitis and irritated skin by topical administration of a composition comprising at least one of sunflower oil and non-saponifiable materials from sunflower oil, wherein the composition stimulates the production of the skin lipids cholesterol, ceramide 1, and ceramide 2. The degree of moisturization of the upper epidermal layers is thereby increased.

US 2003/0170194 A1 discloses a pharmaceutical and/or cosmetic composition containing an organosiloxane and a phospholipid. The composition contains a maximum of 14% by weight of alcohol in order not to cause pain when applied topically to the skin. The document provides a composition which shows good stability. When the composition is applied topically it enters the body within a short period of time.

### OBJECTS OF THE INVENTION

It is an object of the invention to provide a liquid composition for topical administration of a pharmaceutically or cosmetically active agent to the skin of a person or an animal, which is easily administrable.

Another object of the present invention is to provide a liquid composition for topical administration of a pharmaceutically or cosmetically active agent to the skin of a person or an animal, which is capable of forming a coherent lipid layer on the skin.

It is desirable that the aforementioned composition exhibits one or more of the following features upon application to the skin:
reduction of water loss through the skin;
re-establishment of the protective barrier of the skin if applied to skin if said barrier has been compromised;
lack of a feeling of greasiness;
lack of skin irritation.

Another object of the present invention is to provide a liquid composition for topical administration of a disinfectant agent to the skin of a person or an animal, which is easily administrable.

Another object of the present invention is to provide a liquid composition for topical administration of a disinfectant agent to the skin of a person or an animal, which is capable of forming a coherent lipid layer on the skin.

It is desirable that a disinfectant composition exhibits one or more of the following features upon application to the skin:
absence of a dehydrating effect on the skin;
lack of a feeling of greasiness;
lack of skin irritation.

Other objects of the invention include the provision of a pharmaceutical or cosmetic carrier for a pharmaceutically or cosmetically active agent intended for administration to the skin of a person or an animal and a method for incorporating a pharmaceutically or cosmetically active agent into the carrier so as to form a topical pharmaceutical or cosmetic composition of the invention.

Still another object of the present invention is to provide a disinfectant carrier for a disinfectant agent intended for administration to the skin of a person or an animal.

Further objects of the invention will be evident from the following summary of the invention, preferred embodiments thereof described in form of examples, and from the appended claims.

### SUMMARY OF THE INVENTION

According to the present invention there is provided a topical carrier for a topical composition, the carrier comprising 99% by weight or more of phosphatidylcholine and volatile solvent selected from the group consisting of: ethanol; ethanol and C₃- and/or C₄-alcohol; ethanol and volatile silicone oil; ethanol, C₃- and/or C₄-alcohol and volatile silicone oil.

According to the present invention, there is provided a topical carrier comprising 99% by weight or more of phosphatidylcholine and volatile solvent selected from the group consisting of: ethanol; ethanol and C₃- and/or C₄-alcohol; ethanol and volatile silicone oil; ethanol, C₃- and/or C₄-alcohol and volatile silicone oil.

In one embodiment, there is provided a topical carrier comprising from 3% to 15% or 20% or 25% or 30% or 60% by weight of phosphatidylcholine, the remainder being ethanol of a concentration of at least 40% by weight, the ethanol optionally comprising one or several of:
i) up to 20% or 30% or 40% or even up to 50% by weight of C₃-C₄ alcohol;
ii) up to 60% by weight of volatile silicone oil; and
iii) up to 1% by weight of antioxidant, colorant, odorant, and/or preservative; and
iv) up to 2% by weight of denaturant.

It is preferred for the carrier to comprise or to consist of from 5% to 15% or 20% or 25% or 30% or 60% by weight of phosphatidylcholine, the remainder being ethanol in a concentration of at least 50% by weight, the ethanol optionally comprising one or several of:
i) from 2% up to 20% or 30% or 40% by weight of C₃- and/or C₄-alcohol;
ii) from 5% up to 40% or 50% or 60% by weight of volatile silicone oil;
iii) up to 1% by weight of antioxidant, colorant, odorant and/or preservative; and
iv) up to 2% by weight of denaturant.

According to the present disclosure, “vehicle” is synonymous with “carrier”. Unless otherwise stated, percentages given herein are all referring to % by weight.

Examples of C₃-alcohol are n-propanol and 2-propanol (isopropanol).

Examples for C₄-alcohol are 1-butanol, 2-butanol and tert-butanol.

Any component of the volatile solvent has a boiling point of 150° C. or less at ambient pressure (1 atm), except for volatile silicone oil, which may have a boiling point at 1 atm of up to 250° C. Preferred silicone oils have boiling points in the range of 180-250° C. at 1 atm.

A particularly preferred volatile silicone oil is or comprises decamethylcyclopentasiloxane (cyclomethicone 5-NF). Other preferred silicone oils are or comprise dodecamethylcyclohexasiloxane, decamethyltetrasiloxane and/or dodecamethylpentasiloxane.

An antioxidant of the invention is any additional component that inhibits other components from degrading due to oxidation. Antioxidants are exemplified by, but not limited to, reducing agents such as thiols, ascorbic acid, or polyphenols, free radical scavengers such as tocopherols (Vitamin E) and tocotrienols, sequestering agents such as EDTA and phosphonates, or organic acids such as acetic acid, citric acid, glycolic acid or lactic acid.

A person skilled in the art understands which colorants, odorants, and preservatives can be used in a carrier according to the present invention.

A denaturant as defined in this application is an agent or mixture of agents making the cosmetic composition of the invention unattractive for human consumption. Examples of denaturants are esters of phthalic acid, 2-isopropyl-5-methyl-phenol, denatonium benzoate, 3-methyl-cyclopentadecanone, ethyl acetate and their combinations. C₃ and/or C₄ alcohols may be a part of the denaturant system but in the context of the invention they belong to category i) above.

According to the invention there is also provided a topical composition for reducing water loss through the skin, which composition substantially consists of a carrier of the invention.

According to the invention there is also provided a topical composition substantially free of volatile silicone oil.

Phosphatidylcholine of the invention can be natural or synthetic. Natural phosphatidylcholine includes enriched phospholipid from soybeans (soy lecithin, soy-PC, for example Lipoid S 100 and Lipoid S 75), sunflower or rapeseed, containing at least 50% by weight of phosphatidylcholine, the remainder consisting mainly of other polar lipids (such as phosphatidylethanolamine, phosphatidylglycerol, phophatidylinositol and galactolipids) and acylglycerols (monoacylglycerols, diacylglycerols and triacylglycerols). A high content of phosphatidylcholine gives an efficient reduction of water loss, without causing greasiness on the skin. Examples of synthetic phosphatidylcholine comprise dioleoyl phosphatidylcholine and dimyristoyl phosphatidylcholine.

According to the invention there is also provided a topical composition, which substantially consists of:
a) from 90% or 95% or 98% and up to 100% by weight of a topical carrier of the invention; and
b) from 0.001% or 0.1% to 2% or 5% or exceptionally up to 10% by weight of one or more pharmaceutically active agent(s), cosmetically active agent(s) and/or disinfectant agent(s).

According to another embodiment, a topical pharmaceutical, cosmetic or disinfectant composition according to the present invention substantially consists of:

a) from 70% or 80% or 90% or 95% or 98% and up to 100% by weight of a topical carrier of the invention;

b) from 0.001% or 0.1% to 2% or 5% or 10% or 20% or exceptionally up to 30% by weight of one or more pharmaceutically active agent(s), cosmetically active agent(s) and/or disinfectant agent(s).

A topical pharmaceutical, cosmetic or disinfectant composition of the invention can be prepared by dissolving one or more pharmaceutically active agents, cosmetically active agents and/or disinfectant agents respectively, in the carrier or in one or more components of the carrier followed by adding the other components of the carrier and mixing.

At room temperature (20° C.), which is a convenient temperature for administration, the carrier and the compositions of the invention are single-phase homogeneous liquids.

The compositions of the invention are preferably administered to the skin by spraying. For administration any spraying pump suitable for topical administration of liquid compositions can be used. Other preferred means of administration are brushing, dripping, rolling or wiping. Evaporation of the volatile solvent from the skin leaves a coherent layer thereon. The layer so formed lacks a greasy feeling, reduces water loss through the skin, and/or re-establishes the protective skin barrier if compromised.

The compositions of the invention are well tolerated by healthy human skin, and even by persons with sensitive skin, in particular irritated and dry skin.

In one embodiment of the invention, there is provided a topical pharmaceutical composition substantially consisting of:

a) from 90% or 95% or 98% and up to 99.999% by weight of a topical carrier of the invention;

b) from 0.001% or 0.1% to 2% or 5% or exceptionally up to 10% by weight of one or more pharmaceutically active agent(s).

A pharmaceutically active agent comprised by the composition of the invention may be any agent suitable for treating a skin condition amenable to topical treatment.

The one or more pharmaceutically active agent(s) of the invention is selected from the group consisting of: antimicrobial agent, antibiotic; antimycotic agent; antibacterial agent; antifungal agent; antiviral agent; antiseptic; anti-phlogistic; anti-pruritic agent; anti-psoriatic agent; antitussive agent; anti-alopecia agent; anti-acne agent; anti-inflammatory agent; analgesic; antiulcer agent; local anaesthetic; immune response modifying agent. More particularly, the pharmaceutically active agent of the invention is selected from: antibacterial agents, such as oxytetracycline, fusidic acid, gentamycine, mupirocin, retapamulin (and pharmaceutically acceptable salts and derivatives thereof); antimycotic agents, such as nystatin, clotrimazole, miconazole, econazole, ketoconazole, bifonazole, and combinations of imidazole and triazole derivatives, ciclopirox, terbinafine, fluconazole, and amorolfine (and pharmaceutically acceptable salts and derivatives thereof); antiviral agents, such as aciclovir, valaciclovir, penciclovir, famciclovir, foscarnet (trisodium phosphonoformate hexahydrate) and docosanol (and pharmaceutically acceptable salts and derivatives thereof); antiseptics, such as chlorhexidine, benzalkonium chloride and hydrogen peroxide; anti-inflammatory agents (glucocorticoids), such as hydrocortisone, clobetasone, triamcinolone, betamethasone, mometasone, and clobetasol (and pharmaceutically acceptable salts and derivatives thereof); antiphlogistics/analgesics, such as acetylsalicylic acid, salicylic acid, diclofenac, ketoprofen, ibuprofen, naproxen, capsaicin, curcumin, nicotinate (and pharmaceutically acceptable salts and derivatives thereof); antipruritic agents, such as glucocorticoids, for example, hydrocortisone, clobetasone, clobetasol, desonide, mometasone and betamethasone (and pharmaceutically acceptable salts and derivatives thereof) and such as menthol and camphor; antipsoriatic agents, such as calcipotriol, calcitriol, 7-dehydrocholesterol, cholecalciferol, maxacalcitol, doxercalciferol, paricalcitol, inecalcitol, eldecalcitol, betamethasone and cyclosporine A (and pharmaceutically acceptable salts and derivatives thereof); agents for treatment of eczema and atopic dermatitis: tacrolimus and pimecrolimus (and pharmaceutically acceptable salts and derivatives thereof); antiglaucomateous agents, such as timolol, betaxolol, latanoprost, bimatoprost, and travoprost (and pharmaceutically acceptable salts and derivatives thereof); local anaesthetics, such as lidocaine, prilocaine, ropivacaine, mepivacaine, bupivacaine, levobupivacaine, benzocaine, and tetracaine (and pharmaceutically acceptable salts and derivatives thereof); agents for erectile dysfunction, such as alprostadil (prostaglandin E1) (and pharmaceutically acceptable salts and derivatives thereof); anti-dandruff agents, such as selenium sulphides, piroctone oleamine and ketoconazole; anti-alopecia agents, such as minoxidil (and pharmaceutically acceptable salts and derivatives thereof); anti-acne agents, such as tretinoin (retinoic acid), isotretinoin, adapalene, benzoyl peroxide, clindamycin, azelaic acid, niacinamide (and pharmaceutically acceptable salts and derivatives thereof); wound healing agents, such as pantothenic acid, dexpanthenol and fusidic acid (and pharmaceutically acceptable salts and derivatives thereof); steroid hormones, such as prednisone, dexamethasone, triamcinolone, fludrocortisone, testosterone, estradiol, distilbestrol; peptide hormones, such as oxytocin, LL-37, DPK-060 and PXL-01 (and pharmaceutically acceptable salts and derivatives thereof).

According to a another embodiment of the invention, a topical pharmaceutical composition of the invention comprising or consisting of:

a) from 90% or 95% or 98% and up to 99.999% by weight of a topical carrier of the invention consisting of:

from 3% or 5% to 15% or 20% or 25% or 30% or 60% of phosphatidylcholine, the remainder being ethanol of a concentration of at least 40%, the ethanol optionally comprising one or several of:
i) up to 20% or 30% or 40% or even up to 50% of C₃-C₄ alcohol;
ii) up to 60% of volatile silicone oil, in particular of decamethylcyclopentasiloxane;
iii) up to 1% of antioxidant, colorant, odorant and/or preservative; and
iv) up to 2% of denaturant; and

b) up to 10% of pharmaceutically active agent(s);

wherein the weight portions of carrier (a) and at least one pharmaceutically active agent(s) (b) in the composition are adding up to 100%.

According to another embodiment, the topical pharmaceutical composition comprises 5% to 25% of phosphatidylcholine, 50% to 90% of ethanol, up to 40% of volatile silicone oil and up to 10% of pharmaceutically active agent(s).

Particularly preferred pharmaceutically active agents are hydrocortisone (or esters thereof), betamethasone (or esters therof), mometasone furoate, diclofenac (or salts thereof) and/or calcipotriol.

A pharmaceutical composition of the invention is intended to efficiently deliver the active agent into the skin. The compositions are intended and useful for topical treatment, where transdermal passage of the active ingredient is minimized or avoided. Thus, the pharmaceutical composition is neither intended nor useful for transdermal delivery of a pharmaceutically active agent.

Pharmaceutical compositions of the invention are particularly useful for treating inflammatory conditions, such as atopic dermatitis. Hydrocortisone is a preferred pharmaceutically active agent for treating erythema that can be incorporated into the carrier of the invention and can be comprised by the composition of the invention. Diclofenac is another preferred pharmaceutically active agent for treating inflammation of the skin that can be incorporated into the carrier of the invention and can be comprised by the composition of the invention.

Pharmaceutical compositions of the invention are also particularly useful for treating psoriasis. Calcipotriol is a preferred pharmaceutically active agent for treating psoriasis that can be incorporated into the carrier of the invention and can be comprised by the composition of the invention as a single pharmaceutically active agent or in combination with other pharmaceutically active agents such as corticosteroids.

According to an embodiment, the topical cosmetic composition substantially consists of a carrier of the present invention.

According to an embodiment, there is provided a topical cosmetic composition of the invention substantially consisting of:

a) from 90% or 95% or 98% and up to 99.999% by weight of a topical carrier of the invention; and

b) from 0.001% or 0.1% to 2% or 5% or exceptionally up to 10% by weight of one or more cosmetically active agent(s).

A cosmetically active agent comprised by the composition of the invention may be any agent suitable for cosmetic use.

The one or more cosmetically active agent(s) of the invention is selected from the group consisting of antiperspirants, such as aluminium chlorohydrate; sun screens, such as avobenzone, bemotrizinol, diethylamino hydroxybenzoyl hexyl benzoate (Uvinul A Plus), 2-ethylhexyl methoxycinnamate (octinoxate), 2-ethylhexyl 2-hydroxybenzoate (octisalate), octocrylene, oxybenzone ; tanning agents, such as dihydroxyacetone; insects repellants, such as Deet; keratolytics, such as glycolic acid, lactic acid, malic acid, salicylic acid, allantoin, urea and sulphur; antidandruff agents; cooling agents, such as menthol and camphor; glidants; moisturizing agents, such as glycerol, sorbitol, propylene glycol, butandiols, pentanediols, hexanediols, dexpanthenol, urea and lactic acid. Urea is a preferred keratolytic agent, which can be incorporated into the topical carrier of the invention in an amount of up to 10% by weight of the total composition.

According to an embodiment, the topical cosmetic composition of the present invention comprises 5% to 25% of phosphatidylcholine, 50% to 90% of ethanol, up to 40% of volatile silicone oil as carrier; and up to 10% of cosmetically active agent(s).

Particularly preferred cosmetically active agents are urea, dexpanthenol, glycolic acid and lactic acid.

A cosmetic composition of the invention is well tolerated, even by persons with sensitive skin, in particular irritated and dry skin.

According to a another embodiment of the invention, there is provided a topical cosmetic composition of the invention consisting of:

a) from 90% or 95% or 98% and up to 99.999% by weight of a topical carrier of the invention consisting of:

from 3% or 5% to 15% o r 20% o r 25% or 30% or 60% of phosphatidylcholine, the remainder being ethanol of a concentration of at least 40%, the ethanol optionally comprising one or several of:
i) up to 20% or 30% or 40% or even up to 50% of C₃-C₄ alcohol;
ii) up to 60% of volatile silicone oil, in particular of decamethylcyclopentasiloxane;
iii) up to 1% of antioxidant, colorant, odorant and/or preservative; and
iv) up to 2% of denaturant. and

b) up to 10% of cosmetically active agent(s);

wherein the weight portions of carrier (a) and at least one cosmetically active agent(s) (b) in the composition are adding up to 100%.

According to an embodiment, there is provided a topical disinfectant composition of the present invention substantially consisting of a carrier of the present invention.

According to an embodiment, there is provided a disinfectant composition of the invention comprising:

a) from 90% or 95% or 98% and up to 99.999% by weight of the topical carrier of the invention; and

b) from 0.001% or 0.1% to 2% or 5% or exceptionally up to 10% by weight of one or more disinfectant agent(s).

By a disinfectant agent is meant any agent with antibacterial, antifungal and/or antiviral activity.

A disinfectant composition according to the present invention is capable of forming a thin coherent layer on the skin.

The one or more disinfectant agent(s) of the invention is selected from the group consisting of cationic amines, such as benzalkonium chloride and chlorhexidine; organic acids, such as lactic acid, citric acid and lauric acid; and diols, such as propylene glycol, butandiols, pentanediols, hexanediols, and octanediols.

According to a another embodiment of the invention, there is provided a topical disinfectant composition of the invention consists of:

a) from 90% or 95% or 98% and up to 99.999% by weight of a topical carrier of the invention consisting of:

from 3% or 5% to 15% or 20% or 25% or 30% or 60% of phosphatidylcholine, the remainder being ethanol of a concentration of at least 40%, the ethanol optionally comprising one or several of:
i) up to 20% or 30% or 40% or even up to 50% of C₃-C₄ alcohol;
ii) up to 60% of volatile silicone oil, in particular of decamethylcyclopentasiloxane;
iii) up to 1% of antioxidant, colorant, odorant and/or preservative; and
iv) up to 2% of denaturant:

and

b) up to 10% of disinfectant agent(s);

wherein the weight portions of carrier (a) and at least one disinfectant agent(s) (b) in the composition are adding up to 100%.

According to an embodiment of the present invention, the topical disinfectant composition comprises 3% to 15% of phosphatidylcholine, 70% to 95% of ethanol and up to 10% of disinfectant agent(s).

Particularly preferred disinfectant agents are chlorhexidine, lactic acid, propylene glycol and octanediols.

According to the present invention, there is provided a spraying device comprising a composition according to the present invention, optionally comprising a propellant.

### DESCRIPTION OF PREFERRED EMBODIMENTS

Material and Methods

TABLE 1
Materials used for the compositions of the examples.

| Material | Trade name | Supplier | CAS No. |
|---|---|---|---|
| Isopropyl | | Aldrich | 110-27-0 |
| myristate | | | |
| Medium chain | Capmul MCM C8 | Abitec | 26402-26-6 |
| monoglycerides | EP | Corporation | |
| Phosphatidyl- | Soybean lecithin, | Lipoid GmbH | 8002-43-5 |
| choline (>94%) | Lipoid S 100 | | |
| Phosphatidyl- | Soybean lecithin, | Lipoid GmbH | 8002-43-5 |
| choline (>68%) | Lipoid S 75 | | |
| Phosphatidyl- | Soybean lecithin, | Lipoid GmbH | 8002-43-5 |
| choline | Lipoid S 45 | | |
| (approx. 45%) | | | |
| Phosphatidyl- | Sunflower lecithin | Lipoid GmbH | 8002-43-5 |
| choline (>90%) | Lipoid H 100 | | |
| Phosphatidyl- | Sunflower lecithin | Lipoid GmbH | 8002-43-5 |
| choline (>50%) | Lipoid H 50 | | |
| Phosphatidyl- | Rapeseed lecithin | Lipoid GmbH | 8002-43-5 |
| choline (>90%) | Lipoid R 100 | | |
| Ethanol 99.9% | | VWR | 64-17-5 |
| 2-propanol | | Rathburn | 67-63-0 |
| Tert-butanol | | Sigma-Aldrich | 75-65-0 |
| Ethyl acetate | | Rathburn | 141-78-6 |
| Decamethyl- | Cyclomethicone | Dow Corning | 541-02-6 |
| cyclopentasiloxane | 5-NF | | |
| Decamethyl- | DC Fluid 345 | Dow Corning | 541-02-6 |
| cyclopentasiloxane | | | |
| Decamethyl- | | Sigma-Aldrich | 141-62-8 |
| tetrasiloxane | | | |
| Hexamethyl- | DC 200 Fluid, 0.65 | Dow Corning | 107-46-0 |
| disiloxane | CST | | |

Pharmacologically active agents, cosmetically active agents, disinfectant agents and excipients used in the formulation experiments (with CAS Nos) were acetic acid (64-19-7), benzalkonium chloride (8001-54-5), benzoyl peroxide (94-36-0), betamethasone dipropionate (5593-20-4), butylhydroxytoluene (128-37-0), calcipotriol (112965-21-6), camphor (76-22-2), capsaicin (404-86-4), chlorhexidine (55-56-1), cholesterol (57-88-5), citric acid (77-92-9), clindamycin hydrochloride (21462-39-5), curcumin (458-37-7), dexpanthenol (81-13-0), diclofenac sodium (15307-79-6), diethylamino hydroxybenzoyl hexyl benzoate (Uvinul A Plus, 302776-68-7), econazole nitrate (24169-02-6), estradiol (50-28-2), glycerol (56-81-5), glycolic acid (79-14-1), hydrocortisone (50-23-7), hydrocortisone acetate (50-03-3), hydrocortisone butyrate (13609-67-1), ketoprofen (22071-15-4), lactic acid (50-21-5), lauric acid (143-07-7), lidocaine (137-58-6), menthol (1490-04-6), minoxidil (38304-91-5), mometasone furoate (83919-23-7), mupirocin (12650-69-0), naproxen (22204-53-1), niacinamide (98-92-0), octinoxate (5466-77-3), octisalate (118-60-5), oxytocin acetate (50-56-6), prilocaine (721-50-6), propylene glycol (57-55-6), sodium fusidate (751-94-0), tacrolimus (104987-11-3), terbinafine hydrochloride (78628-80-5) and urea (57-13-6). Lidocaine and prilocaine were from Moehs (Spain) and all other substances from Sigma-Aldrich.

The effect on human skin of prior art pharmaceutical compositions and of carriers and compositions of the invention was observed by determining transepidermal water loss and skin oiliness by using DermaLab Combo equipment (Cortex Technology, Denmark).

Transepidermal water loss (TEWL) indicates the skin's ability to retain water, i.e. its barrier function on transepidermal water loss. The probe used for TEWL measurement consists of an open-chamber with two combined humidity/temperature sensors mounted in a cylindrical diffusion chamber (10 mm diameter). After application of the probe onto the skin, the TEWL value is recorded when the standard deviation of the measure values has stabilized at less than 0.2 units (typically at 30-45 seconds).

Skin oiliness measurement is related to the feeling of greasiness of a formulation after application. The oiliness can be assessed visually or measured by sampling oil from the surface of the skin by pressing a tape (Sebutape, CuDerm Corporation, U.S.A.) onto the skin for a few seconds. The grey Sebutape becomes black upon contact with oils and the change in color is measured with Dermalab equipment.

### EXAMPLE 1

Determination of TEWL at 30 and 90 minutes and skin oiliness after application of various compositions not comprised by the invention (petrolatum and A-B) and compositions according to the invention (C-G).

Changes in skin barrier function were determined after a single application of petrolatum and compositions A through G to the skin of healthy volunteers (Table 2).

Rectangular areas (6 cm²) were marked on the volar parts of the left forearms of ten healthy male persons. The respective composition (12 pl) was evenly distributed on the test area.

TEWL was measured at 30 and 90 minutes after application of the compositions and compared to petrolatum (vaseline, a common ointment base) and a non-treated area. The occlusive effect of petrolatum decreased the TEWL value as compositions nos. C-G did. Compositions nos. A and B gave TEWL values slightly higher than the non-treated area. These results indicate that a significantly improved barrier against TEWL is obtained by applying phosphatidylcholine containing compositions to the skin.

Oily residues on the skin were measured 90 minutes after application by sampling of the surface with Sebutape. Petrolatum gave the highest value while compositions A and B gave higher values than that of the non-treated area. Composition C gave lower value than the non-treated area. Compositions E and F gave slightly higher values than the non-treated area but significantly lower values than petrolatum and compositions A and B.

The presence of urea (compositions D and G) increased the oiliness compared to the same compositions without urea (C and F). These data indicate that non-greasy lipid compositions can be formed by phosphatidylcholine containing compositions even in the presence of the volatile silicone oil cyclomethicone 5-NF or urea.

TABLE 2
TEWL and skin oiliness after application of various carriers and compositions

| | Carrier or composition | | | | | | | | |
|---|---|---|---|---|---|---|---|---|---|
| | Not | | | | | | | | |
| | treated | Petrolatum** | A** | B** | C* | D* | E* | F* | G* |
| Components | Components, % by weight | | | | | | | | |
| Isopropyl myristate | | | 10.2 | 25 | | | | | |
| Medium chain | | | 10 | 25 | | | | | |
| monoglycerides | | | | | | | | | |
| Phosphatidylcholine | | | | | 20 | 20.2 | 20 | 50 | 49.9 |
| Ethanol | | | 79.8 | 50 | 80 | 74.8 | 20 | 50 | 45.1 |
| Urea | | | | | | 5.0 | | | 5.0 |
| Cyclomethicone 5- | | | | | | | 60 | | |
| NF | | | | | | | | | |
| TEWL 30 min | 5.2 | 3.9 | 5.5 | 5.5 | 4.6 | 3.8 | 4.3 | 3.6 | 3.3 |
| TEWL 90 min | 4.7 | 4.1 | 5.4 | 5.9 | 3.9 | 4.4 | 4.3 | 3.7 | 3.5 |
| ΔTEWL 30 min | — | −1.4 | 0.3 | 0.2 | −0.6 | −1.5 | −1.0 | −1.6 | −2.0 |
| ΔTEWL 90 min | — | −0.6 | 0.7 | 1.2 | −0.8 | −0.3 | −0.4 | −1.0 | −1.2 |
| Skin oiliness | 1.3 | 23.1 | 7.0 | 7.5 | 0.3 | 5.0 | 2.2 | 2.9 | 8.2 |

*Composition or carrier of the invention.
**Prior art carrier or composition and carrier or composition not comprised by the invention

The compositions of Table 2 were prepared according to the following general procedure. The components were weighed and dissolved in ethanol. If needed, short ultrasonication and/or gentle heating were applied until a clear liquid had been formed. In composition E the clear liquid was diluted with silicone oil. The final products were stored in air-tight glass vials at ambient temperature.

### EXAMPLE 2

### Pharmaceutical and Disinfectant Compositions of the Invention

Twentyfive examples of the topical pharmaceutical compositions and two disinfectant compositions of the invention are listed in Table 3 and 4. They were prepared by adding a pre-weighed amount of the respective pharmaceutically active agent or disinfectant agent to one of the carriers of Example 1. The mixtures were gently heated and ultrasonicated until clear solutions had been formed.

TABLE 3
Examples of pharmaceutical compositions of the invention

| Composi- | Carrier | Carrier, | Pharmacologically | Active agent, |
|---|---|---|---|---|
| tion # | # | % by weight | active agent | % by weight |
| Pharm-1 | C | 99.4 | Benzalkonium chloride | 0.6 |
| Pharm-2 | C | 99.0 | Benzoyl peroxide | 1.0 |
| Pharm-3 | C | 99.9 | Betamethasone | 0.1 |
| | | | dipropionate | |
| Pharm-4 | C | 99.1 | Hydrocortisone | 0.9 |
| | | | butyrate | |
| Pharm-5 | F | 99.1 | Chlorhexidine | 0.9 |
| Pharm-6 | E | 95.6 | Diclofenac sodium | 1.4 |
| Pharm-7 | F | 97.2 | Diclofenac sodium | 4.4 |
| Pharm-8 | F | 98.9 | Estradiol | 1.1 |
| Pharm-9 | F | 98.8 | Minoxidil | 1.2 |
| Pharm-10 | F | 99.0 | Mupirocin | 1.0 |
| Pharm-11 | E | 99.9 | Tacrolimus | 0.1 |
| Pharm-12 | C | 98.2 | Econazol nitrate | 1.8 |
| Pharm-13 | C | 99.0 | Oxytocin acetate | 1.0 |
| Pharm-14 | E | 96.0 | Lidocaine | 4.0 |
| Pharm-15 | C | 91.7 | Lidocaine + Prilocaine | 4.4 + 3.9 |
| Pharm-16 | F | 99.0 | Sodium fusidate | 1.0 |
| Pharm-17 | E | 99.99 | Calcipotriol | 0.005 |
| Pharm-18 | F | 99.99 | Calcipotriol | 0.005 |
| Pharm-19 | F | 98.7 | Hydrocortisone | 1.3 |
| Pharm-20 | C | 98.3 | Hydrocortisone acetate | 1.7 |
| Pharm-21 | C | 99.9 | Mometasone furcate | 0.1 |
| Pharm-22 | C | 99.0 | Clindamycin | 1.0 |
| | | | hydrochloride | |
| Pharm-23 | F | 99.83 | Curcumin | 0.17 |
| Pharm-24 | C | 99.29 | Capsaicin | 0.71 |
| Pharm-25 | C | 98 | Menthol + Camphor | 1.0 + 1.0 |

TABLE 4
Examples of disinfectant compositions of the invention

| Composi- | Carrier | Carrier, | Disinfectant | Active agent, |
|---|---|---|---|---|
| tion # | # | % by weight | agent | % by weight |
| Dis-1 | C | 99.4 | Benzalkonium | 0.6 |
| | | | chloride | |
| Dis-2 | F | 99.1 | Chlorhexidine | 0.9 |

### EXAMPLE 3

Further examples of carriers and compositions of the invention. The examples of carriers and compositions listed in Tables 5-8 were prepared according to the procedures outlined in Example 1 and 2.

TABLE 5
Examples of carriers of the invention

| Component | Carrier #* | | | | | | | | | |
|---|---|---|---|---|---|---|---|---|---|---|
| (% w/w) | H-1 | H-2 | H-3 | R-1 | S-1 | S-2 | S-3 | S-4 | S-5 | S-6 |
| Lipoid S 100 | | | | | | 15.1 | 15.0 | 14.9 | 5.1 | 19.4 |
| Lipoid R 100 | | | | 19.9 | | | | | | |
| Lipoid H 100 | 20.5 | 20.6 | 10.8 | | | | | | | |
| Lipoid S 75 | | | | | 14.7 | | | | | |
| Lipoid H 50 | | | 9.6 | | | | | | | |
| Ethanol 99.9% | 21.0 | 71.5 | 71.6 | 24.4 | 9.6 | 9.8 | 47.8 | 84.9 | 94.9 | 29.1 |
| 2-propanol | | 6.4 | 6.4 | | | | | | | |
| Tert-butanol | | 0.8 | 0.8 | | | | | | | |
| Ethyl acetate | | 0.8 | 0.8 | | | | | | | |
| DC Fluid 345 | | | | | 75.6 | 75.1 | 37.3 | | | |
| Cyclomethicone | 58.5 | | | 55.7 | | | | | | |
| 5-NF | | | | | | | | | | |
| Decamethyltetra- | | | | | | | | | | 51.6 |
| siloxane | | | | | | | | | | |

*H = based on sunflower lecithin, R = based on rapeseed lecithin, S = based on soybean lecithin

TABLE 6
Examples of pharmaceutical compositions of the invention

| | Composition # | | | | | | | | | |
|---|---|---|---|---|---|---|---|---|---|---|
| Component | Pharm- | Pharm- | Pharm- | Pharm- | Pharm- | Pharm- | Pharm- | Pharm- | Pharm- | Pharm- |
| (% w/w) | 26 | 27 | 28 | 29 | 30 | 31 | 32 | 33 | 34 | 35 |
| Dexpanthenol | 3.4 | | | | | | | | | |
| Tacrolimus | | 1.0 | 1.0 | 1.0 | 1.0 | | | | | |
| Mometason | | | | | | 0.1 | | | | |
| furoate | | | | | | | | | | |
| Clindamycin | | | | | | | 0.9 | | | |
| HCl | | | | | | | | | | |
| Terbinafine | | | | | | | | 1.0 | | |
| HCl | | | | | | | | | | |
| Ketoprofen | | | | | | | | | 2.5 | |
| Niacinamide | | | | | | | | | | 4.5 |
| Lipoid S 100 | 12.7 | 19.8 | 20.1 | 19.9 | 20.0 | 20.0 | 19.4 | 15.1 | 15.0 | 15.8 |
| Citric acid | | 0.5 | | 0.3 | 0.2 | | | | | |
| Acetic acid | | | | | | 0.5 | | | | |
| Butylhydroxy | | | | | | 0.03 | | | | |
| toluene | | | | | | | | | | |
| Ethanol | 19.0 | 78.7 | 78.9 | 78.8 | 78.8 | 79.4 | 27.8 | 84.9 | 61.0 | 79.7 |
| 99.9% | | | | | | | | | | |
| Cyclomethicone | 65.0 | | | | | | 52.0 | | 21.5 | |
| 5-NF | | | | | | | | | | |

TABLE 7
Examples of cosmetic compositions of the invention

| | | (% | Lipoid | Ethanol | | |
|---|---|---|---|---|---|---|
| Composition # | Active(s) | w/w) | S 100 | 99.9% | Other solvent | (% w/w) |
| Cosm-1 | Urea | 5.0 | 19.8 | 73.1 | | |
| | Glycolic | 2.1 | | | | |
| | acid | | | | | |
| Cosm-2 | Urea | 3.3 | 19.9 | 66.8 | Cyclomethicone | 10.0 |
| | | | | | 5-NF | |
| Cosm-3 | Urea | 2.5 | 20.4 | 74.7 | | |
| | Propylene | 2.4 | | | | |
| | glycol | | | | | |
| Cosm-4 | Propylene | 4.9 | 20.3 | 74.8 | | |
| | glycol | | | | | |
| Cosm-5 | Urea | 2.5 | 20.0 | 75.0 | | |
| | Propylene | 2.5 | | | | |
| | glycol | | | | | |
| Cosm-6 | Propylene | 5.1 | 20.0 | 74.9 | | |
| | glycol | | | | | |
| Cosm-7 | Urea | 5.0 | 5.1 | 74.8 | | |
| | Propylene | 15.2 | | | | |
| | glycol | | | | | |
| Cosm-8 | Urea | 4.8 | 4.8 | 69.9 | | |
| | Propylene | 20.4 | | | | |
| | glycol | | | | | |
| Cosm-9 | Urea | 5.0 | 9.6 | 65.6 | | |
| | Propylene | 19.8 | | | | |
| | glycol | | | | | |
| Cosm-10 | Urea | 5.0 | 18.7 | 57.0 | | |
| | Propylene | 19.4 | | | | |
| | glycol | | | | | |
| Cosm-11 | Urea | 5.0 | 10.0 | 75.6 | 2-propanol | 8.5 |
| | | | | | Tert-butanol | 0.9 |
| Cosm-12 | Propylene | 10.0 | 10.0 | 71.2 | 2-propanol | 8.0 |
| | glycol | | | | Tert-butanol | 0.8 |
| Cosm-13 | Glycerol 85% | 10.4 | 10.0 | 70.9 | 2-propanol | 8.0 |
| | | | | | Tert-butanol | 0.8 |
| Cosm-14 | Urea | 3.2 | 10.0 | 70.9 | 2-propanol | 8.0 |
| | Lactic acid | 5.0 | | | Tert-butanol | 0.8 |
| | Glycerol | 2.2 | | | | |
| Cosm-15 | Octinoxate | 9.0 | 8.0 | 63.2 | 2-propanol | 8.0 |
| | Uninul A | 6.0 | | | Tert-butanol | 0.8 |
| | Plus | 5.0 | | | | |
| | Octsalate | | | | | |

TABLE 8
Disinfectant compositions of the invention

| | Composition # | | | | |
|---|---|---|---|---|---|
| Component (% w/w) | Dis-1 | Dis-2 | Dis-3 | Dis-4 | Dis-5 |
| Lactic acid | | 2.48 | 2.5 | 2.5 | |
| Lauric acid | | 2.48 | | | |
| Glycerol, 85% | | 0.50 | 0.52 | | |
| Lipoid S 100 | 4.98 | 11.91 | 6.0 | 6.0 | 6.0 |
| Ethanol 99.9% | 85.52 | 73.51 | 79.9 | 83.2 | 85.4 |
| 2-propanol | 7.60 | 8.26 | 9.0 | 6.7 | 6.8 |
| Tert-butanol | 0.95 | 0.83 | 0.90 | 0.83 | 0.86 |
| Ethyl acetate | 0.95 | | 1.2 | 0.83 | 0.86 |

### EXAMPLE 4

Determination of TEWL at 30 and 90 minutes after application of various compositions not comprised by the invention (Comp-1, -2 and -3) and compositions according to the invention (S-1 to S-5).

TABLE 9
Comparative compositions not comprised by the invention

| | Composition # | | |
|---|---|---|---|
| Component (% w/w) | Comp-1 | Comp-2 | Comp-3 |
| Lipoid S 45 | | 11.9 | |
| Lipoid S 75 | | | 7.5 |
| MCM | 9.3 | | 7.6 |
| Cholesterol | 1.0 | | |
| Ethanol 99.9% | 10.4 | 1.7 | 19.8 |
| 2-propanol | | 9.4 | |
| DC Fluid 345 | 79.3 | | 65.1 |
| Hexamethyldisiloxane | | 77.1 | |

TABLE 10
TEWL after application of various compositions

| | TEWL | | | ΔTEWL | |
|---|---|---|---|---|---|
| Composition | Baseline | 30 min | 90 min | 30 min | 90 min |
| Untreated control | 5.7 | 5.4 | 5.3 | −0.3 | −0.4 |
| Comp-1** | 5.1 | 3.9 | 4.2 | −1.2 | −0.9 |
| Comp-2** | 4.7 | 3.6 | 3.9 | −1.1 | −0.8 |
| Comp-3** | 4.5 | 3.9 | 4.2 | −0.6 | −0.3 |
| S-1* | 4.6 | 2.8 | 2.5 | −1.8 | −2.1 |
| S-2* | 5.5 | 3.4 | 3.6 | −2.0 | −1.9 |
| S-3* | 6.6 | 3.9 | 4.5 | −2.6 | −2.1 |
| S-4* | 6.7 | 4.2 | 4.9 | −2.5 | −1.8 |
| S-5* | 5.1 | 3.8 | 3.9 | −1.3 | −1.2 |

*Compositions of the invention.
**Prior art compositions and compositions not comprised by the invention

Changes in skin barrier function were determined after a single application of Comp-1, -2 and -3 (see Table 9) and S-1 to S-5 (see Table 5) to the skin of healthy volunteers.

Circular areas (3.5 cm²) were marked on the volar parts of the forearms of healthy male and female persons. The respective composition (10 μl) was evenly distributed on the test area.

TEWL was measured before (baseline) and at 30 and 90 minutes after application of the compositions using an untreated area as control. The results are shown in Table 10. All of compositions S-1 to S-5 gave a stronger decrease in TEWL than the comparative compositions (Comp-1 to Comp-3) after 30 minutes. After 90 minutes, the difference in decrease for TEWL of the compositions according to the invention compared to the comparative compositions is even larger, indicating that the barrier reinforcement effect also last longer for compositions according to the invention.

### EXAMPLE 5

### Dehydration, Antibacterial and Antiviral Effect of Disinfectant Compositions

When tested on healthy volunteers it was noted that none of the disinfectant compositions Dis-1, Dis-2, Dis-3, Dis-4 or Dis-5 gave a dehydrating effect on the skin after repeated use, despite their high ethanol content.

Screening tests for antibacterial and antiviral activity were performed using E. Coli bacteria and polio virus respectively, for the compositions Dis-1 and Dis-2. The test results indicate that both products are likely to fulfill the criteria for hand disinfectants stipulated in the EN 1500 and EN 14476 standards.

1. Topical carrier comprising 99% by weight or more of phosphatidylcholine and volatile solvent selected from the group consisting of:
ethanol; ethanol and C₃- and/or C₄-alcohol; ethanol and volatile silicone oil;
ethanol, C₃- and/or C₄-alcohol and volatile silicone oil.

2. The carrier of claim 1, comprising from 3% to 15% or 20% or 25% or 30% or 60% by weight of phosphatidylcholine, the remainder being ethanol of a concentration of at least 40% by weight, the ethanol optionally comprising one or several of:
i) up to 20% or 30% or 40% or even up to 50% by weight of C₃-C₄ alcohol;
ii) up to 60% % by weight of volatile silicone oil;
iii) up to 1% by weight of antioxidant, colorant, odorant, and/or preservative; and
iv) up to 2% by weight of denaturant.

3. The carrier of claim 1, comprising from 5% to 15% or 20% or 25% or 30% or 60% by weight of phosphatidylcholine, the remainder being ethanol in a concentration of at least 50% by weight, the ethanol optionally comprising one or several of:
i) from 2% up to 20% or 30% or 40% or even up to 50% by weight of C₃- and/or C₄-alcohol;
ii) from 5% up to 40% or 50% or 60% by weight of volatile silicone oil;
iii) up to 1% by weight of antioxidant, colorant, odorant and/or preservative; and
iv) up to 2% by weight of denaturant.

4. The carrier according to claim 1, wherein the volatile silicone oil is or comprises decamethylcyclopentasiloxane.

5. Topical composition for reducing water loss through the skin, substantially consisting of the carrier according to claim 1.

6. Topical composition substantially consisting of:
a) from 90% or 95% or 98% and up to 100% by weight of a topical carrier according to claim 1; and
b) from 0.001% or 0.1% to 2% or 5% or exceptionally up to 10% by weight of one or more of pharmaceutically active agent(s), cosmetically active agent(s) and/or disinfectant agent(s).

7. Topical pharmaceutical composition substantially consisting of:
a) from 90% or 95% or 98% and up to 99.999% by weight of a carrier according to claim 1; and
b) from 0.001% or 0.1% to 2% or 5% or exceptionally up to 10% by weight of one or more pharmaceutically active agent(s).

8. The pharmaceutical composition according to claim 7, which comprises 5% to 25% of phosphatidylcholine, 50% to 90% of ethanol, up to 40% of volatile silicone oil and up to 10% of pharmaceutically active agent(s).

9. The composition according to claim 7, wherein the pharmaceutically active agent(s) is selected from the group consisting of: antibacterial agents, such as oxytetracycline, fusidic acid, gentamycine, mupirocin, retapamulin (and pharmaceutically acceptable salts and derivatives thereof); antimycotic agents, such as nystatin, clotrimazole, miconazole, econazole, ketoconazole, bifonazole, and combinations of imidazole and triazole derivatives, ciclopirox, terbinafine, fluconazole, and amorolfine (and pharmaceutically acceptable salts and derivatives thereof); antiviral agents, such as aciclovir, valaciclovir, penciclo vir, famciclovir, foscarnet (trisodium phosphonoformate hexahydrate) and docosanol (and pharmaceutically acceptable salts and derivatives thereof); antiseptics, such as chlorhexidine, benzalkonium chloride and hydrogen peroxide; anti-inflammatory agents (glucocorticoids), such as hydrocortisone, clobetasone, triamcinolone, betamethasone, mometasone, and clobetasol (and pharmaceutically acceptable salts and derivatives thereof); antiphlogistics/analgesics, such as acetylsalicylic acid, salicylic acid, diclofenac, ketoprofen, ibuprofen, naproxen, capsaicin, nicotinate (and pharmaceutically acceptable salts and derivatives thereof); antipruritic agents, such as glucocorticoids, for example, hydrocortisone, clobetasone, clobetasol, desonide, mometasone and betamethasone, and local anaesthetics, for example, lidocaine and prilocaine (and pharmaceutically acceptable salts and derivatives thereof); antipsoriatic agents, such as calcipotriol, calcitriol, 7-dehydrocholesterol, cholecalciferol, maxacalcitol, doxercalciferol, paricalcitol, inecalcitol, eldecalcitol, betamethasone and cyclosporine A (and pharmaceutically acceptable salts and derivatives thereof); agents for treatment of eczema and atopic dermatitis: tacrolimus and pimecrolimus (and pharmaceutically acceptable salts and derivatives thereof); antiglaucomateous agents, such as timolol, betaxolol, latanoprost, bimatoprost, and travoprost (and pharmaceutically acceptable salts and derivatives thereof); local anaesthetics, such as lidocaine, prilocaine, ropivacaine, mepivacaine, bupivacaine, levobupivacaine, benzocaine, and tetracaine (and pharmaceutically acceptable salts and derivatives thereof); agents for erectile dysfunction, such as alprostadil (prostaglandin E1) (and pharmaceutically acceptable salts and derivatives thereof); anti-dandruff agents, such as selenium sulphides, piroctone oleamine and ketoconazole; anti-alopecia agents, such as minoxidil (and pharmaceutically acceptable salts and derivatives thereof); anti-acne agents, such as tretinoin (retinoic acid), isotretinoin, adapalene, benzoyl peroxide, clindamycin, azelaic acid (and pharmaceutically acceptable salts and derivatives thereof); wound healing agents, such as pantothenic acid, dexpanthenol and fusidic acid (and pharmaceutically acceptable salts and derivatives thereof); steroid hormones, such as prednisone, dexamethasone, triamcinolone, fludrocortisone, testosterone, estradiol, distilbestrol; peptide hormones, such as oxytocin, LL-37, DPK-060 and PXL-01 (and pharmaceutically acceptable salts and derivatives thereof).

10. The composition according to claim 7, wherein the pharmaceutically active agent(s) is selected from the group consisting of: hydrocortisone (or esters thereof), betamethasone (or esters therof), mometasone furoate, diclofenac (or salts thereof) and/or calcipotriol.

11. Topical cosmetic composition substantially consisting of a carrier according to claim 1.

12. Topical cosmetic composition, comprising:
a) from 90% or 95% or 98% and up to 99.999% by weight of a carrier according to claim 1; and
b) from 0.001% or 0.1% to 2% or 5% or exceptionally up to 10% by weight of one or more cosmetically active agent(s).

13. The cosmetic composition according to claim 11,
wherein the one or more cosmetically active agent(s) are selected from the group consisting of: antiperspirants such as aluminium chlorohydrate; sun screens, such as avobenzone, bemotrizinol, diethylamino hydroxybenzoyl hexyl benzoate, octisalate, octocrylene, oxybenzone ; tanning agents, such as dihydroxyacetone; insects repellants, such as Deet; keratolytics, such as glycolic acid, lactic acid, malic acid, salicylic acid, allantoin, urea and sulfur; antidandruff agents; glidants; moisturizing agents, such as glycerol, sorbitol, propylene glycol, butanediol, pentanediol, hexanediol, dexpanthenol, urea, lactic acid.

14. The cosmetic composition according to claim 11, wherein the one or more cosmetically active agent(s) are selected from the group consisting of: urea, dexpanthenol, glycolic acid and lactic acid.

15. Topical disinfectant composition substantially consisting of a carrier according to claim 1.

16. Topical disinfectant composition, comprising:
a) from 90% or 95% or 98% and up to 99.999% by weight of a carrier according to claim 1; and
b) from 0.001% or 0.1% to 2% or 5% or exceptionally up to 10% by weight of one or more disinfectant agent(s).

17. The composition according to claim 15, wherein the one or more disinfectant agents are selected from the group consisting of: cationic amines, such as benzalkonium chloride and chlorhexidine; organic acids, such as lactic acid, citric acid and lauric acid; and diols, such as propylene glycol, butandiols, pentanediols, hexanediols, and octanediols.

18. The composition according to claim 15, wherein the one or more disinfectant agent(s) are selected from the group consisting of:
chlorhexidine, lactic acid, propylene glycol and octanediols.

19. Spraying device comprising a composition according to claim 5, optionally comprising a propellant.
